(19) United States
(12) Patent Application Publication
(10) Pub. No.: US 2025/0213631 A1
(43) Pub. Date: Jul. 3, 2025

(54) Cardiovascular Health-Promoting Composition

(71) Applicant: John Charles Davidson, Calgary (CA)

(72) Inventor: John Charles Davidson, Calgary (CA)

(21) Appl. No.: 19/085,320

(22) Filed: Mar. 20, 2025

Related U.S. Application Data
(62) Division of application No. 18/158,543, filed on Jan. 24, 2023.
(60) Provisional application No. 63/322,431, filed on Mar. 22, 2022.

(51) Int. Cl. A61K 35/747; A61K 31/575; A61K 36/81; A61P 3/06

(52) U.S. Cl. CPC A61K 35/747; A61K 31/575; A61K 36/81; A61P 3/06

(57) ABSTRACT

Described herein is a composition comprising a unique and novel combination of natural ingredients supporting cardiovascular health by contributing to healthy serum cholesterol levels while also reducing CRP and IL-6 cytokine levels. Furthermore, the composition promotes the development of a healthy or healthier gut microbiome. The composition comprises 10%-70% Lactobacillus plantarum, Lactobacillus reuteri and mixtures thereof; 2%-86% plant sterols; and 4%-88% Ashwagandha root powder.

### PRIOR APPLICATION INFORMATION

The instant application is a divisional application of U.S. patent application Ser. No. 18/158,543, filed Jan. 24, 2023, which claimed the benefit of U.S. Provisional Patent Application Ser. No. 63/322,431, filed Mar. 22, 2022, now abandoned, and entitled “CARDIOVASCULAR HEALTH-PROMOTING COMPOSITION”, the entire contents of which are incorporated herein by reference for all purposes.

### BACKGROUND OF THE INVENTION

Over 35 million Americans alone take statin drugs to reduce cholesterol, yet some people are still unable to get their cholesterol under control, and heart disease remains the leading killer of Americans.

Probiotics are beneficial bacteria, two strains of which, L. plantarum and L. reuteri have been demonstrated to reduce LDL cholesterol levels and reduce C-reactive protein (CRP). For example, studies have shown that these probiotics promote favorable outcomes in hypercholesterolemic and/or hyperlipidemic patients, including statistically significant reductions in LDL level and cardiovascular events.

Plant sterols are plant components that have a chemical structure similar to cholesterol except for the addition of an extra methyl or ethyl group. However, despite the structural similarity, plant sterol absorption in humans is considerably less than that of cholesterol. In fact, there is evidence that plant sterols reduce cholesterol absorption and thus reduce circulating levels of cholesterol.

Withania somnifera, also known as ashwagandha or winter cherry is sold in many countries as a medicinal herb or as a dietary supplement. Typically, ashwagandha is prepared as a root powder. Ashwagandha is used in traditional medicines to decrease inflammation, cortisol-related anxiety and cholesterol.

### SUMMARY OF THE INVENTION

According to a first aspect of the invention, there is provided a long-term storage stable composition comprising:
10%-70% Lactobacillus, selected from the group consisting of: Lactobacillus plantarum, Lactobacillus reuteri and mixtures thereof;
2%-86% plant sterols; and
4%-88% Ashwagandha root powder.

In some embodiments of the invention, the composition is storage-stable for at least 6 months.

In some embodiments, the probiotic microorganism is a mixture of Lactobacillus plantarum and Lactobacillus reuteri.

In some embodiments, the Lactobacillus plantarum is one or more of CECT 7527 (Lactobacillus plantarum probiotic strain), CECT 7528 (Lactobacillus plantarum probiotic strain), and CETC 7529 (Lactobacillus plantarum probiotic strain).

In some embodiments, the Lactobacillus plantarum and/or Lactobacillus reuteri is added at 1×10⁶, 1×10⁷, 1×10⁸, 1×10⁹, 1×10¹⁰ or more colony forming units (CFU) per gram.

In some embodiments, the composition has at least 1×10⁹ CFU per gram after 6 months.

In some embodiments, the plant sterols are a mixture of β-sitosterol, campesterol and stigmasterol.

In some embodiments, the mixture of plant sterols is at least 35% β-sitosterol, at least 20% campesterol and at least 10% stigmasterol.

In some embodiments, the mixture of plant sterols is at least 40% β-sitosterol, at least 26% campesterol and at least 18% stigmasterol.

In some embodiments, the storage-stable composition comprises (a) 100 mg Lactobacillus plantarum, Lactobacillus reuteri or mixtures thereof; (b) 50 mg Ashwagandha root powder; and (c) 370 mg plant sterols.

According to another aspect of the invention, there is provided a method of treating or ameliorating one or more symptoms of hypercholesterolemia and/or hyperlipidemia in an individual suffering from hypercholesterolemia and/or hyperlipidemia and/or diagnosed with high LDL cholersterol, said method comprising:
administering to the individual an effective amount of a composition comprising:
   10%-70% Lactobacillus, selected from the group consisting of: Lactobacillus plantarum, Lactobacillus reuteri and mixtures thereof;
   2%-86% plant sterols; and
   4%-88% Ashwagandha root powder.

According to another aspect of the invention, there is provided use of the composition as described above for treating, reducing the severity of or ameliorating hypercholesterolemia and/or hyperlipidemia.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a composition comprising a unique and novel combination of natural ingredients supporting cardiovascular health by contributing to healthy serum cholesterol levels while also reducing CRP and IL-6 cytokine levels. Furthermore, the composition promotes the development of a healthy or healthier gut microbiome.

According to the invention, there is provided a storage-stable composition comprising (a) a probiotic microorganism; (b) an Ashwagandha extract; and (c) plant sterols.

In some embodiments of the invention, the probiotic microorganism is a Lactobacillus, for example, Lactobacillus plantarum or Lactobacillus reuteri.

In some embodiments of the invention, probiotic microorganism is a mixture of Lactobacillus plantarum and Lactobacillus reuteri.

In some embodiments of the invention, the Lactobacillus plantarum is one or more of CECT 7527, CECT 7528 and CETC 7529.

As is known by those of skill in the art, at least some strains of L. plantarum reduce systolic blood pressure; and leptin, LDL cholesterol and fibrinogen concentrations (Naruszewicz et al., 2002, Am J Clin Nutrition 76:1249-1255). Additionally, some strains of Lactobacillus have been shown to be anti-atherogenetic and/or to reduce the arteriosclerotic index of patients (Ding et al., 2017, Oncotarget 8:59915-59928).

Furthermore, Lactobacilli are associated with the production of short chain fatty acids, which are associated with better colon health.

In some embodiments of the invention, the Lactobacillus plantarum and/or Lactobacillus reuteri is added at 1×10⁶, 1×10⁷, 1×10⁸, 1×10⁹, 1×10¹⁰ or more colony forming units (CFU) per gram.

In some embodiments of the invention, the Ashwagandha is a root powder of Ashwagandha. It is noted that such root powder extracts are available commercially. For example, in some methods, the Ashwagandha roots are cut off or otherwise separated from the Ashwagandha plant, cleaned to remove any contaminants, infected roots or adhered dust or the like, pulverized and then separated over size 10 MESH to 30 MESH. It Is noted that such preparations of Ashwagandha root powder are known in the art and are commercially available. For example, such commercial preparations are typically classified by activity, for example, Ashwagandha Root PE 40:1 std. 5% by HPLC gives 5% active with anolides.

Similarly, there are several commercially available mixtures of plant sterols, for example, mixtures that have for example β-sitosterol, campesterol or stigmasterol as their main ingredient. In some embodiments of the invention, the plant sterols or phytosterols are a mixture of β-sitosterol, campesterol and stigmasterol, for example, a mixture of plant sterols that is at least 35% for example at least 40% β-sitosterol, at least 20%, for example, at least 25% or at least 26% campesterol and at least 10%, for example, at least 15% or at least 18% stigmasterol.

In some embodiments of the invention, the storage-stable composition comprises:
10%-70% probiotic microorganism, selected from the group consisting of: Lactobacillus plantarum, Lactobacillus reuteri and mixtures thereof;
2%-86% plant sterols;
4%-88% Ashwagandha root powder.

In some embodiments of the invention, the long-term storage stable composition comprises:
10-25% Lactobacillus, selected from the group consisting of: Lactobacillus plantarum, Lactobacillus reuteri and mixtures thereof;
50-86% plant sterols; and
4-20% Ashwagandha root powder.

In some embodiments of the invention, the probiotic microorganism, the plant sterols and the Ashwagandha root powder are mixed together as dry powders and distributed to capsules, for example, 100 mg capsules, 150 mg capsules, 200 mg capsule, 250 mg capsules, 300 mg capsules, 350 mg capsule, 400 mg capsules, 450 mg capsules, 500 mg capsules, 550 mg capsules, 600 mg capsules, 650 mg capsules, 700 mg capsules or 750 mg capsules.

In some embodiments of the invention, non-medicinal additives such as for example magnesium stearate and/or microcrystalline cellulose and the like may be added to the mixture prior to distribution into capsules.

In some embodiments, suitable pharmaceutically acceptable excipients may be added to the storage-stable composition.

In other embodiments of the invention, the storage-stable composition comprises (a) 50-125 mg Lactobacillus; (b) 20-100 mg Ashwagandha root powder; and (c) 250-425 mg plant sterols. In these embodiments, the storage-stable composition is in a capsule that is at least 500 mg.

In some embodiments of the invention, the storage-stable composition comprises (a) 100 mg Lactobacillus; (b) 50 mg Ashwagandha root powder; and (c) 370 mg plant sterols. In these embodiments, the storage-stable composition is in a capsule that is at least 500 mg.

For example, in these embodiments, the composition may be in the form of a 520 mg capsule, a 525 mg capsule, a 550 mg capsule or a 600 mg capsule.

In some embodiments, the capsules are sprayed with an enteric coating. However, in other embodiments, the capsule may already be impregnated with a suitable coating prior to distribution of the mixture therein. As will be appreciated by one of skill in the art, a suitable coating or an enteric coating is a coating such that when the coated capsule reaches the alkaline environment of the lower bowel, the coating on the capsule dissolves so that the contents of the capsule are released.

In some embodiments of the invention, the composition is administered daily for a period of at least 12 weeks.

In some embodiments of the invention, the composition is administered to an individual who is also being administered statins, for example, a statin drug on a dosage regimen or schedule.

The statin may be a statin drug selected from the group consisting of atorvastatin, Fluvastatin, lovastatin, pitavastatin, pravastatin, rosuvastatin, and simvastatin.

In some embodiments of the invention, there is provided use of the composition of the invention for treating, reducing the severity of or ameliorating hypercholesterolemia.

According to another embodiment of the invention, there is provided a method of treating, ameliorating and/or reducing the severity of one or more symptoms associated with hypercholesterolemia and/or hyperlipidemia, for example, high LDL levels, chest pain and/or high blood pressure, comprising administering an effective amount of the composition of the invention to an individual in need of such treatment, for example, an individual suffering from or diagnosed with high LDL cholesterol and/or hypercholesterolemia.

As per above, the composition may be administered to the individual or patient or human patient on a dosage regimen of daily administration for at least 12 weeks.

It is noted that LDL cholesterol levels of 130-159 mg/dl are considered to be “borderline high”, while levels of 160-189 mg/dl are considered to be “high” and 190 mg/dl and above is considered to be very high.

As discussed herein, use of the composition and/or administration on a dosage regimen as discussed above will result in one or more of the following: increased levels of gut or microbiome flora associated with good health, that is, a more favorable gut flora; better intestinal/gastrointestinal health; reduction of LDL cholesterol levels; improvement of total cholesterol levels; increased energy; and reduction of fatigue.

As discussed herein, it is surprising that a mixture of: Lactobacillus plantarum and/or Lactobacillus reuteri with Ashwagandha root powder can retain activity for an extended period of time, for example, at least 1 month, at least 2 months, at least 3 months, at least 4 months, at least 5 months or at least 6 months or more.

Specifically, Ashwagandha is known to have antibacterial properties. For example, Bisht and Rawat demonstrated that Ashwagandha had anti-microbial properties against many Gram-positive bacteria, include methicillin resistant Staphylococcus and Enterococcus. Accordingly, one of skill in the art would have anticipated that a mixture of Ashwagandha root powder with a probiotic such as Lactobacillus plantarum and/or Lactobacillus reuteri would result in the antibacterial activity of the Ashwagandha root powder at least inhibiting growth of the probiotics.

Furthermore, probiotics have an extremely low water activity (Aw), for example, above 0.95. However, powders such as Ashwagandha root powder and phytosterols are typically produced at much lower Aw. As is known to those of skill in the art, blending powders having different Aw can affect the stability of such a mixture.

As discussed herein, “storage-stable” refers to the fact that, contrary to expectations, the number of Lactobacillus bacteria capable of forming colonies (colony-forming units) after 6 months was greater than 10% of the initial number of bacterial CFU added to the composition. For example, if the initial bacterial CFU per gram added to the composition is 1×10¹⁰, after 6 months, the bacterial CFU per gram of the composition will be at least 1×10⁹.

While the probiotic strains L. planetarium and L. reuteri alone are efficacious ingredients capable of reducing LDL cholesterol, the addition of ashwagandha promotes reduction of other cardiovascular factors, such as, for example, triglyceride levels, CRP, IL-6, cortisol and body fat levels. As such, the surprising stability of the composition indicates that individuals in need thereof can be administered the composition and receive the benefits of the synergistic actions of the Ashwagandha root and the Lactobacillus as discussed herein. For example, the composition may be administered on a dosage regimen, for example, as a daily dosage, wherein “daily” does not necessarily mean “every day” but may mean for example 13 out of 14 days, 9 out of 10 days, 6 out of 7 days, 5 out of 7 days, 3 out of 4 days or 2 out of 3 days.

As discussed herein, the mechanism responsible for the cholesterol-lowering effect of the composition is the inhibition of intestinal cholesterol absorption in the liver due to increased bile salt hydrolase activity. Specifically, this enzyme hydrolyzes bile salts into amino residues and free bile salts and suppresses bile acid reabsorption.

The invention will now be further explained and/or elucidated by way of example. However, the invention is not necessarily limited to or by the examples.

### EXAMPLE 1

### Long-Term Stability Test

Table 1 shows the results of a 6-month stability test.

Specifically, Formulation (A) is a control, comprising 100 mg of Lactobacillus and 400 mg of microcrystalline cellulose; Formulation (B) comprises 100 mg of Lactobacillus, 370 mg of phytosterols and 30 mg of microcrystalline cellulose; and Formulation (C) comprises 100 mg Lactobacillus, 370 mg phytosterols, 50 mg Ashwagandha root powder and 30 mg microcrystalline cellulose.

As indicated in Table 1, the source of Lactobacillus is Floradapt™; however, other sources of suitable Lactobacillus strains may be used within the invention.

The respective mixtures were

As can be seen, the composition of the three components (Formulation C) basically showed the same trend as probiotics alone (Formulation A). Specifically, the number of viable probiotic cells in the blend with the phytosterols and Ashwagandha trends similar to the control, so these ingredients are a compatible match. That is, each of the formulation preparations initially had 1.3-2.0 ×10¹⁰ bacterial cells. As can be seen in Table 1, at 1 month, 3 months and 6 months, the number of viable bacterial cells in each of the three formulation preparations are approximately equivalent, never dropping below 5×10⁹ cfu per gram. This indicates that, contrary to expectations, the presence of the Ashwagandha root powder did not reduce the bacterial counts in formulation preparation (C) and there were no instability issues in either formulation preparation (B) or (C) despite the blending of powders having different Aw. As such, the number of Lactobacillus bacteria capable of forming colonies (colony-forming units) after 6 months was greater than 10% of the initial number of bacterial CFU added to the composition.

While the preferred embodiments of the invention have been described above, it will be recognized and understood that various modifications may be made therein, and the appended claims are intended to cover all such modifications which may fall within the spirit and scope of the invention.

TABLE 1
Results of Stability test

| | | Theoretical | | | | |
|---|---|---|---|---|---|---|
| | | viable cell | | | | |
| | Water | counts based | | | | |
| | Activity at | on the CoA of | Viable Cell Counts (cfu/g) | | | |
| Blend | the time of | probiotics | T = 0 (Initial) | T = 1 month | T = 3 month | T = 6 month |
| Formulation | preparation | bulk | Mar. 21, 2021 | Apr. 19, 2021 | Jun. 21, 2021 | Sep. 17, 2021 |
| A) Floradapt | 0.048 @ | 10.2E+10 | 1.30E+10 | 1.20E+10 | 7.40E+09 | 5.20E+09 |
| Cardio + | 25° C. | | | 92% | 57% | 40% |
| MCC (control) | | | | | | |
| B) Floradapt | 0.093 @ | 10.2E+10 | 1.40E+10 | 2.20E+10 | 9.40E+09 | 4.50E+09 |
| Cardio + | 25° C. | | | 157% | 67% | 32% |
| Phytosterols + | | | | | | |
| MCC | | | | | | |
| C) Floradapt | 0.124 @ | 10.1E+10 | 2.00E+10 | 4.20E+10 | 5.70E+10 | 5.00E+09 |
| Cardio + | 25° C. | | | 210% | 285% | 25% |
| Phytosterols + | | | | | | |
| Ashwagandha + | | | | | | |
| MCC | | | | | | |

1. A method of treating or ameliorating one or more symptoms of hypercholesterolemia and/or hyperlipidemia in an individual suffering from hypercholesterolemia and/or hyperlipidemia and/or diagnosed with high LDL cholersterol, said method comprising:
administering to the individual an effective amount of a composition comprising:
10%-70% (w/w) Lactobacillus, selected from the group consisting of: Lactobacillus plantarum, Lactobacillus reuteri and mixtures thereof;
2%-86% (w/w) plant sterols; and
4%-88% (w/w) Ashwagandha root powder.

2. The method according to claim 1 wherein the composition is administered daily for a period of at least 12 weeks.

3. The method according to claim 1 wherein the individual is also being administered a statin drug on a dosage regimen or schedule.

4. The method according to claim 3 wherein the statin drug is selected from the group consisting of atorvastatin, Fluvastatin, lovastatin, pitavastatin, pravastatin, rosuvastatin, and simvastatin.

5. The method according to claim 1 wherein the Lactobacillus is a mixture of Lactobacillus plantarum and Lactobacillus reuteri.

6. The method according to claim 1 wherein the Lactobacillus plantarum is one or more strain selected from the group consisting of CECT 7527 (Lactobacillus plantarum probiotic strain), CECT 7528 (Lactobacillus plantarum probiotic strain) and CETC 7529 (Lactobacillus plantarum probiotic strain).

7. The method according to claim 1 wherein the plant sterols are a mixture of β-sitosterol, campesterol and stigmasterol.

8. The method according to claim 7 wherein the mixture of plant sterols is at least 35% (w/w) β-sitosterol, at least 20% (w/w) campesterol and at least 10% (w/w) stigmasterol.

9. The method according to claim 7 wherein the mixture of plant sterols is at least 40% (w/w) β-sitosterol, at least 26% (w/w) campesterol and at least 18% (w/w) stigmasterol.

10. The method according to claim 1 wherein the composition consists essentially of
10-25% (w/w) Lactobacillus, selected from the group consisting of: Lactobacillus plantarum, Lactobacillus reuteri and mixtures thereof;
50-86% (w/w) plant sterols comprising at least 35% (w/w) β-sitosterol, at least 20% (w/w) campesterol and at least 10% (w/w) stigmasterol; and
4-20% (w/w) Ashwagandha root powder.

11. The method according to claim 1 wherein the effective amount of the composition consists essentially of (a) 50-125 mg Lactobacillus; (b) 20-100 mg Ashwagandha root powder; and (c) 250-425 mg plant sterols comprising at least 35% (w/w) β-sitosterol, at least 20% (w/w) campesterol and at least 10% (w/w) stigmasterol; in a 500 mg or 600 mg capsule.

12. The method according to claim 1 wherein the effective amount of the composition comprises (a) 100 mg Lactobacillus plantarum, Lactobacillus reuteri or mixtures thereof; (b) 50 mg Ashwagandha root powder; and (c) 370 mg plant sterols comprising at least 35% (w/w) β-sitosterol, at least 20% (w/w) campesterol and at least 10% (w/w) stigmasterol; in a 500 mg or 600 mg capsule.

13. The method according to claim 1 wherein the composition consists essentially of
10-25% (w/w) Lactobacillus plantarum, selected from the group consisting of one or more of Lactobacillus plantarum CECT 7527, Lactobacillus plantarum CECT 7528 and Lactobacillus plantarum CETC 7529; Lactobacillus reuteri, or mixtures thereof;
50-86% (w/w) plant sterols comprising at least 35% (w/w) β-sitosterol, at least 20% (w/w) campesterol and at least 10% (w/w) stigmasterol; and
4-20% (w/w) Ashwagandha root powder.

14. The method according to claim 1 wherein the effective amount of the composition consists essentially of:
(a) 50-125 mg Lactobacillus plantarum, selected from the group consisting of one or more of Lactobacillus plantarum CECT 7527, Lactobacillus plantarum CECT 7528 and Lactobacillus plantarum CETC 7529; Lactobacillus reuteri or mixtures thereof;
(b) 20-100 mg Ashwagandha root powder; and
(c) 250-425 mg plant sterols comprising at least 35% (w/w) β-sitosterol, at least 20% (w/w) campesterol and at least 10% (w/w) stigmasterol in a 500 mg or 600 mg capsule.

15. The method according to claim 1 wherein the composition consists essentially of
(a) 100 mg Lactobacillus plantarum, selected from the group consisting of one or more of Lactobacillus plantarum CECT 7527, Lactobacillus plantarum CECT 7528 and Lactobacillus plantarum CETC 7529; Lactobacillus reuteri, or mixtures thereof;
(b) 50 mg Ashwagandha root powder; and
(c) 370 mg plant sterols the mixture of plant sterols is at least 35% (w/w) β-sitosterol, at least 20% (w/w) campesterol and at least 10% (w/w) stigmasterol; in a 500 mg or 600 mg capsule.

16. The method according to claim 1 wherein the composition is in the form of a coated capsule, said coated capsule being coated such that when the coated capsule reaches the alkaline environment of the lower bowel, the coating on the coated capsule dissolves so that the contents of the capsule are released.

17. The method according to claim 1 wherein the composition is administered daily for a period of at least 12 weeks.

18. The method according to claim 17 wherein the daily administration for a period of at least 12 weeks results in one or more of the following: increased levels of gut or microbiome flora associated with good health, that is, a more favorable gut flora; better intestinal/gastrointestinal health; reduction of LDL cholesterol levels; improvement of total cholesterol levels; increased energy; and reduction of fatigue.

19. The method according to claim 1 wherein the individual in need of such treatment has LDL cholesterol levels of 130-159 mg/dL.
